(19) United States
(12) Patent Application Publication
(10) Pub. No.: US 2009/0312748 A1
(43) Pub. Date: Dec. 17, 2009

(54) Rotational detachment mechanism

(76) Inventors: Kirk L. Johnson, Weston, FL (US); David A. Kilmer, North Miami, FL (US); Juan A. Lorenzo, Davie, FL (US); Robert Lulo, Pembroke Pines, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/157,550

(22) Filed: Jun. 11, 2008

(51) Int. Cl. A61B 17/00

(52) U.S. Cl. 606/1

(57) ABSTRACT

A handle for use with an implantable medical device deployment system that guides and deploys the medical device at a target location within a body vessel. The handle includes a rotatable member, rotation of which causes movement of a control member of the deployment system in an axial direction. An actuator in a handle may include a threaded portion which has at least two different pitch sizes.

### CROSS-REFERENCE TO RELATED APPLICATION

This patent application is a continuation which claims the benefit of (i) U.S. Provisional Patent Application No. 60/749,784, filed Dec. 13, 2005, and PCT/US2006/61916 filed Dec. 12, 2006; and (ii) U.S. Provisional Patent Application No. 60/749,838, filed Dec. 13, 2005 and PCT/US2006/61925 filed Dec. 12, 2006, which are hereby incorporated herein by reference.

### BACKGROUND AND SUMMARY OF THE INVENTION

This invention generally relates to handles to having rotational detachment mechanisms for use with medical device deployment systems that deploy implantable medical devices at target locations within a human body vessel, and methods of using the same.

### DESCRIPTION OF RELATED ART

The use of catheter delivery systems for positioning and deploying therapeutic devices, such as dilation balloons, stents and embolic coils, in the vasculature of the human body has become a standard procedure for treating endovascular diseases. It has been found that such devices are particularly useful in treating areas where traditional operational procedures are impossible or pose a great risk to the patient, for example in the treatment of aneurysms in cranial blood vessels. Due to the delicate tissue surrounding cranial blood vessels, especially for example brain tissue, it is very difficult and often risky to perform surgical procedures to treat defects of the cranial blood vessels. Advancements in catheter deployment systems have provided an alternative treatment in such cases. Some of the advantages of catheter delivery systems are that they provide methods for treating blood vessels by an approach that has been found to reduce the risk of trauma to the surrounding tissue, and they also allow for treatment of blood vessels that in the past would have been considered inoperable.

Typically, these procedures involve inserting the distal end of a delivery catheter into the vasculature of a patient and guiding it through the vasculature to a predetermined delivery site. An implantable medical device, such as an embolic coil or vascular stent, is attached to the end of a delivery member which pushes the medical device through the catheter and out of the distal end of the catheter into the delivery site. Some of the delivery systems associated with these procedures utilize an elongated control member, sometimes referred to as a control wire or pull wire, to activate the release and deployment of the medical device. For example, U.S. Pat. No. 5,250,071 to Palermo, which is hereby incorporated herein by reference, describes a delivery and detachment system whereby interlocking clasps of the system and the coil are held together by a control wire. The control wire is moved proximally to disengage the clasps from each other.

Additionally, U.S. patent application Ser. No. 11/461,245, filed Jul. 31, 2006, to Mitelberg, et al., which is hereby incorporated herein by reference for its disclosure of a distal-portion detachment mechanism with which the present invention may be utilized, describes a detachment system wherein a control wire engages a hook or an eyelet to attach a medical device to the deployment system. The control wire is moved in a proximal direction to disengage it from the hook and release the medical device.

There remains a need for mechanisms or methods that may be used by a medical professional to manipulate control members of various medical device deployment systems. There also remains a need for mechanisms or methods that reduce the strain on the control member, while providing a quick and timely deployment of the implantable medical device at a target location within a body vessel.

### SUMMARY OF THE INVENTION

In accordance with one embodiment or aspect of the present invention, a handle is provided for use with an implantable medical device deployment system including a control member whose movement initiates the release of an implantable medical device from the deployment system. The handle includes a handle body, with a cavity having a rotatable member located within the cavity that rotates relative to the handle body. The rotatable member includes an internal threaded surface defining a lumen, wherein the internal threaded surface may be threadably connected to the control member and the lumen may receive the control member therein. The rotatable member is rotated to cause the control member to move axially and release the implantable medical device.

Alternatively, relative rotational movement between the rotatable member and the handle body can cause the handle body to move in an axial direction relative to a carrier member. The handle body may be operatively connected to the control member so that the control member moves in an axial direction with the handle body to release the medical device.

In accordance with yet another embodiment or aspect of the present invention, a deployment system is provided for delivering an implantable medical device to a target location of a body vessel. The deployment system comprises a generally elongated carrier member having a proximal end portion and a distal end portion, and an implantable medical device releasably attached to the distal end portion of the carrier member. The deployment system also includes a control member whose movement causes the release of the implantable medical device from the distal end portion of the carrier member. Additionally, the deployment system includes a handle having a handle body connected to the distal end portion of the carrier member. The handle body has a cavity in which a rotatable member is located. The rotatable member is rotatable relative to the handle body. Furthermore, the rotatable member includes an internal threaded surface defining a lumen having a proximal end portion of the control member located therein and threadably engaged with the internal threaded surface of rotatable member. The rotatable member is rotated to cause the control member to move axially to release the implantable medical device.

In accordance with a further embodiment or aspect of the present invention, a deployment system may also include a handle that has a rotatable member and a handle body. The rotatable member is rotationally coupled to the distal end portion of the carrier member and includes an internal threaded surface defining a lumen. The handle body has a threaded portion located within the lumen of the rotatable member and threadably engaged with the internal threaded surface of the rotatable member. The handle body is operatively connected to the control member so that the control member moves with the handle body. Either the rotatable member or the handle body may be rotated to cause the handle body to move in an axial direction relative to the carrier member, thereby causing movement of the control member to release the medical device.

In accordance with another embodiment or aspect of the present invention, an actuator is provided for use with an implantable medical device deployment system that includes a control member which initiates the release of an implantable medical device from the deployment system upon movement of the control member. The actuator comprises an actuator body which may be operatively connected to the control member. The actuator body includes a threaded portion that may be threadably connected to the deployment system so that rotational movement of the actuator body causes the actuator and the control member connected therewith to move relative to the deployment system to release the medical device. The threaded portion of the actuator body includes a pitch that has at least a first pitch size and a second pitch size wherein the first and second pitch sizes are different from each other.

In accordance with further embodiment or aspect of the present invention, a deployment system for delivering an implantable medical device to a target location of a body vessel is provided. The deployment system comprises a generally elongated carrier member having a proximal end portion and a distal end portion and an implantable medical device releasably attached to the distal end portion of the carrier member. The deployment system also includes a control member whose movement causes the release of the implantable medical device from the distal end portion of the carrier member. Additionally, the deployment system also includes an actuator having a threaded portion that is threadably connected to a corresponding threaded portion of the carrier member located at the proximal end portion of the carrier member. The control member is connected to the actuator, and rotational movement of the actuator causes the actuator to move in an axial direction relative to the carrier member, thereby causing movement of the control member in an axial direction to release the medical device form the distal end portion of the carrier member. Further, one of the threaded portion of the actuator and the threaded portion of the carrier member includes a pitch that has at least a first pitch size and a second pitch size wherein the first pitch size and said second pitch size are different from each other.

In accordance with yet another embodiment or aspect of the present invention, a deployment system delivers an implantable medical device to a target location of a body vessel. The deployment system comprises a generally elongated carrier member having a proximal end portion and a distal end portion and an implantable medical device releasably attached to the distal end portion of the carrier member. The deployment system also includes a control member whose movement causes the release of the implantable medical device from the distal end portion of the carrier member. Additionally, the deployment system includes an actuator movably connected to the proximal end portion of the carrier member wherein the actuator is moveable in an axial direction relative to the carrier member. The control member is connected to the actuator, and movement of the actuator relative to the carrier member causes movement of the control member to release the medical device from the distal end portion of the carrier member. Further, the deployment system includes a regulator that changes the rate of relative axial movement between the actuator and the carrier member.

In accordance with another embodiment or aspect of the present invention, a method is provided for deploying an implantable medical device to a target location of a body vessel. The method comprises providing a deployment system that has a generally elongated carrier member having a proximal end portion and a distal end portion and an implantable medical device releasably secured to the distal end portion of the carrier member. The deployment system also has a control member whose movement causes the release of the implantable medical device from the distal end portion of the carrier member. Additionally, the deployment system also has a handle that has a handle body connected to the distal end portion of the carrier member. The handle body includes a cavity having a rotatable member located within the cavity. The rotatable member includes an internal threaded surface defining a lumen having a proximal end portion of the control member located within the lumen and threadably engaged with the internal threaded surface of rotatable member. Rotation of rotatable member causes the control member to move axially. The method further includes positioning the implantable medical device generally adjacent to a target location with a body vessel, and rotating the rotatable member to cause the control member to move axially, thereby releasing the medical device.

In accordance with a further embodiment or aspect of the present invention, a method is provided for deploying an implantable medical device to a target location of a body vessel. The method comprises providing a deployment system including a carrier member having a proximal end portion and a distal end portion and an implantable medical device releasably connected to the distal end portion of the carrier member. The medical device is released from the distal end portion of the carrier member upon movement of a control member. The deployment system also includes an actuator threadably connected to the proximal end portion of the carrier member. The actuator includes a thread that has at least two different pitch sizes. The control member is connected to and moveable with the actuator. The method further comprises positioning the implantable medical device generally adjacent to a target location within the body vessel. Rotating the actuator relative to the carrier member to cause axial movement of the actuator relative to the carrier member, thereby moving the control member and releasing the implantable medical device.

### BRIEF DESCRIPTION OF THE DRAWINGS

FIG. 1 is a cross-sectional view of a medical device deployment system utilizing one embodiment of a handle in accordance with the present invention;

FIG. 2 is a front perspective view of a distal end portion of the carrier member of FIG. 1;

FIG. 3 is a cross-sectional view of the deployment system of FIG. 1, shown after the medical device has been deployed;

FIG. 4 is a top view of the handle shown in FIG. 1;

FIG. 5 is a cross-sectional view of a medical device deployment system utilizing another embodiment of a handle in accordance with the present invention;

FIG. 6 is a cross-sectional view of the deployment system of FIG. 5 shown after the medical device has been deployed;

FIG. 7 is a cross-sectional view of an implantable medical device deployment system utilizing one embodiment of an actuator in accordance with the present invention;

FIG. 8 is a front perspective view of the distal end of the carrier member of FIG. 7 with portions broken away to show the engagement member; and

FIG. 9 is a cross-sectional view of the deployment system of FIG. 7, shown with the actuator in the actuated position.

### DESCRIPTION OF THE PREFERRED EMBODIMENTS

The following description of the preferred embodiments of the present invention is merely illustrative in nature, and as such it does not limit in any way the present invention, its application, or uses. Numerous modifications may be made by those skilled in the art without departing from the true spirit and scope of the invention.

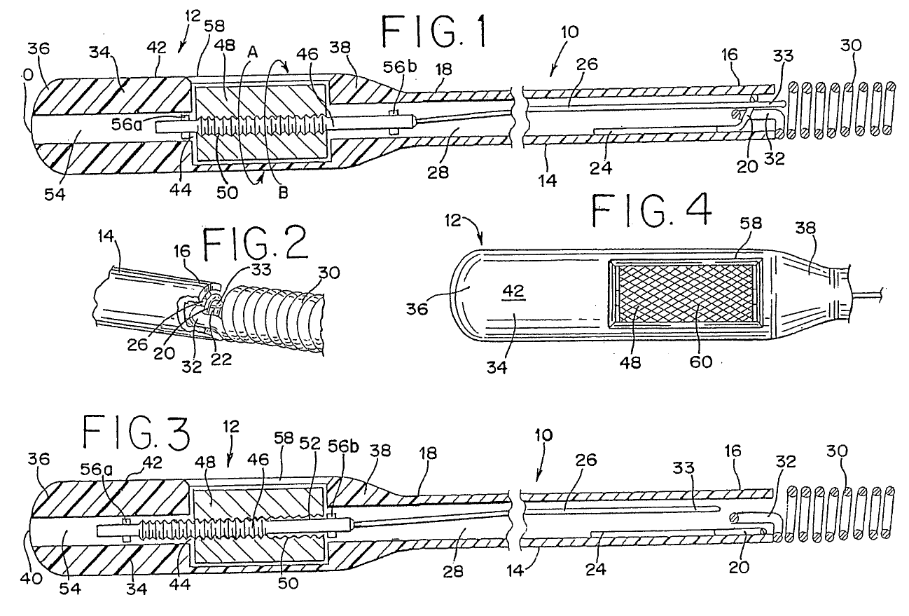

FIGS. 1, 2 and 3 illustrate an implantable medical device deployment system, generally designated at 10, showing one embodiment of a distal end portion deployment system suitable for use with a handle in accordance with the present invention, generally designated at 12. The illustrated deployment system 10 is generally similar to the deployment system disclosed in U.S. patent application Ser. No. 11/461,245, filed Jul. 31, 2006, to Mitelberg et al., which is hereby incorporated herein by reference. However, it will be understood that the handle of the present invention may be used in conjunction with various types of deployment systems having various configurations, features and attachment and release mechanisms, such as the deployment system disclosed in U.S. Pat. No. 5,250,071, which is hereby incorporated herein by reference.

The deployment system 10 is comprised of a generally hollow elongated carrier member or pusher 14 having a distal end portion 16 and a proximal end portion 18. Preferably, the carrier member 14 is a hypotube that may be comprised of a biocompatible material, such as stainless steel. The hypotube typically will have a diameter of between about 0.010 inch (0.254 mm) and about 0.015 inch (0.381 mm), a preferred tube having a diameter of approximately 0.013 inch (0.330 mm). Such a carrier member 14 is suitable for delivering and deploying implantable medical devices, such as embolic coils, vascular stents or the like, to target locations, typically aneurysms, within the neurovasculature, but differently sized carrier members comprised of other materials may be useful for different applications.

An engagement member 20 is associated with the distal end portion 16 of the carrier member 14. The engagement member 20 may comprise a distal end length of an elongated wire loosely bent in half to define an opening 22 (FIG. 2). The proximal end or ends 24 of the engagement member 20 may be fixedly connected to the carrier member 14 at a location proximal to the distal end portion 16.

In an alternative embodiment, the engagement member 20 may comprise a flat ribbon defining the opening 22 at a distal portion thereof. In either embodiment, the engagement member 20 typically is preferably deformable to the up-turned condition illustrated in FIGS. 1 and 2. Additionally, the engagement member 20 may preferably be elastically deformable to the up-turned condition of FIGS. 1 and 2, such that it will return to a substantially flat condition, (illustrated in FIG. 3) when not otherwise constrained, as will be explained in more detail below. When not elastically deformable, the engagement member up-turned condition is generally straightened by contact with another component that “unbends” this member. The engagement member 20 may be comprised of any of a number of materials, including nitinol and stainless steel. The function of the engagement member 20 will be described in greater detail herein.

The deployment system 10 further includes a control member 26, such as a control wire or pull wire, received within the lumen 28 of the carrier member 14 and movable with respect to the engagement member 20. The control member 26 stretches beyond the proximal end portion 18 of the carrier member 14 and is operatively connected to the handle 12. The control member 26 may be a wire comprised of any of a number of materials, including nitinol. The function of the control member 26 will be described in greater detail herein.

As shown in FIGS. 1 and 2, an implantable medical device 30, such as the illustrated embolic coil, is releasably attached to the distal end portion 16 of the carrier member 14 by the engagement member 20. However, it will be appreciated that virtually any implantable medical device may be delivered and deployed by the deployment system.

To connect the implantable medical device 30 to the distal end portion 16 of the carrier member 14, an aperture-containing proximal end portion 32 of the implantable medical device 30 is placed adjacent to opening 22 of the engagement member 20, which is then deformed to the up-turned condition of FIGS. 1 and 2. Alternatively, the opening 22 may be moved to the up-turned condition prior to placement of the implantable medical device 30. In the up-turned condition, at least a portion of the opening 22 passes through the aperture of the proximal end portion 32.

As described herein, the engagement member 20 may be elastically deformable to the up-turned condition of FIGS. 1 and 2 so it will tend to return to a substantially flat condition as illustrated in FIG. 3. In order to prevent this, and to consequently lock the implantable medical device 30 to the engagement member 20, the distal end portion 33 of the control member 26 is moved axially through the opening 22 to the position shown in FIGS. 1 and 2. In this connected condition, the control member 26 holds the engagement member 20 in the up-turned condition, and the engagement member 20 releasably secures the proximal end portion 32 of the implantable medical device 30 to the distal end portion 16 of the carrier member 14.

The handle 12 can include a handle body 34 having a proximal end portion 36 and a distal end portion 38 wherein the distal end portion 38 is connected to the proximal end portion 18 of the carrier member 14. The handle body 34 may be comprised of a proximal wall 40 and a circumferential wall 42 that define a cavity 44. The cavity 44 communicates with the lumen 28 of the carrier member 14 and accepts a proximal end portion 46 of the control member 26. The circumferential wall 42 may be a continuous arcuate wall which forms a handle body having a generally circular cross-section, such as a generally cylindrically shaped handle body, or the circumferential wall could be comprises of a series of panels or sub-walls which form a handle having a rectangular cross-section.

A rotatable member 48, such as the illustrated generally cylindrical rotational sleeve, is located within a portion of the cavity 44 that is configured to house the rotatable member and allow the rotatable member to rotate relative to the handle body 34, the carrier member 14 and the control member 26. Preferably, the rotatable member 48 may rotate in the direction of “A” or the direction of “B” (FIG. 1), as desired or as required by the application of use.

The rotatable member 48 includes an internal threaded surface 50 (perhaps best shown in FIG. 3) which defines a lumen 52 that accepts the proximal end portion 46 of the control member 26. The proximal end portion 46 of the control member 26 is threaded to correspond to and threadably engage the internal threaded surface 50 of the rotatable member 48. The threading on the proximal end portion 46 of the control member 12 may be any suitable threading, such as a grooved surface or a twisted shape of the proximal end 46 of the control member 26.

As the rotatable member 48 is rotated relative to the control member 26, the threaded engagement between the internal surface 50 of the rotatable member 48 and the proximal end portion 46 of the control member 26 causes the control member to move in a proximal or distal direction depending on the direction of rotational movement of the rotatable member 48. If the rotatable member 48 is rotated such that the control member 26 moves in a proximal direction, the cavity 44 of the handle may include a through port 54 at a location that is proximal the rotatable member. The through port 54 accepts the proximal end portion 46 of the control member 26 as the control member is moved proximally out of the proximal end of the rotatable member 48. To prevent over-threading of the control member 26 or to limit the amount of axial movement of the control member 26 in either the proximal or distal direction, the control member 26 may include stops 56a, 56b that contact the rotatable member 48 and prevent further movement of the control member 26 in a particular direction. For example, as illustrated in FIG. 3, when the stop 56b contacts the rotatable member 48, the stop 56b prevents any further movement of the control member 26 in the proximal direction. The locations of the stops 56a and 56b may be varied along the control member 26 and may be placed at pre-selected locations prior to use so that the control member only moves a pre-determined distance in the axial direction.

The handle body 34 and rotatable member 48 are preferably configured so that the rotatable member 48 may be rotated by hand, but both may also be configured to be rotated by instrument. Referring to FIGS. 1, 3 and 4, the circumferential wall 42 of the handle body 34 may include an aperture or window 58 that extends through the wall 42 to allow access to the rotatable member 48 so that the rotatable member may be rotated. Additionally, referring to referring to FIG. 4, the rotatable member 48 preferably includes an outer surface 60 that is textured or knarled to provide a gripping surface that may be gripped by a finger of a user to rotate the rotatable member 48.

In the illustrated embodiment, the medical device 30 may be attached to the deployment system 10 as described above and as illustrated in FIGS. 1 and 2. When the handle 12 is utilized with this type of deployment system to effectuate the release of the medical device from the deployment system, referring to FIG. 3, a user may access the rotatable member 48 through window 58 to cause rotation of the rotatable member relative to the control member 26. The threaded engagement between the internal surface 50 and the proximal end portion 46 of the control member 26 causes the control member to move proximally relative to the engagement member 20. In the illustrated detachment system for the medical device that is shown in the drawings, this proximal movement proceeds so that the distal end portion 33 of the control member 26 moves out of the opening 22 of the engagement member 20. Once the distal end portion 33 of the control member 26 is moved out of opening 22 of the engagement member 20, the engagement member is free to allow release of the medical device 30. FIG. 3 shows the engagement member 20 returned to its flat configuration, thereby releasing the medical device 30. This return may be due to a bias in the engagement member 20 or by a straightening-type of engagement with the medical device 30 as it separates from the detachment device.

According to one method of delivering the medical device 30, a tubular catheter (not shown) is fed into a body vessel until a distal end thereof is adjacent to a target location. Thereafter, the deployment system 10 and associated implantable medical device 30 are advanced through the catheter, using procedures and techniques known in the art, until the device 30 is itself generally adjacent to the target location. Alternatively, the deployment system 10 and associated device 30 may be pre-loaded in the catheter, with the combination being fed through a body vessel to a target location. Other methods of positioning the implantable medical device 30 generally adjacent to a target location may also be practiced without departing from the scope of the present invention.

To more accurately position the engaged device 30, radiopaque markers (not illustrated) may be attached to the carrier member 14 or the device 30 itself.

When the engaged device 30 has been properly positioned and oriented, the rotatable member 48 is rotated, preferably by hand through window 58 in wall 42 of the handle body, relative to the control member 26. Referring to FIG. 3, as the rotatable member 48 is rotated, the threaded engagement between the internal surface 50 of the rotatable member 48 and the proximal end portion 46 of the control member 26 causes the control member 26 to move in a proximal direction and out of engagement with the engagement member 20. The engagement member 20 is allowed to return to its original substantially flat condition or is moved to a release condition by engagement with another component of the system, thereby disengaging the aperture-containing end portion 32 of the implantable medical device 30 and deploying the medical device 30. The control member 26 may be provided with a radiopaque portion to provide visual feedback to indicate when the device 30 has been released.

When the implantable medical device 30 is disengaged from the engagement member 20, the deployment system 10 may be removed from the patient alone or in conjunction with the catheter.

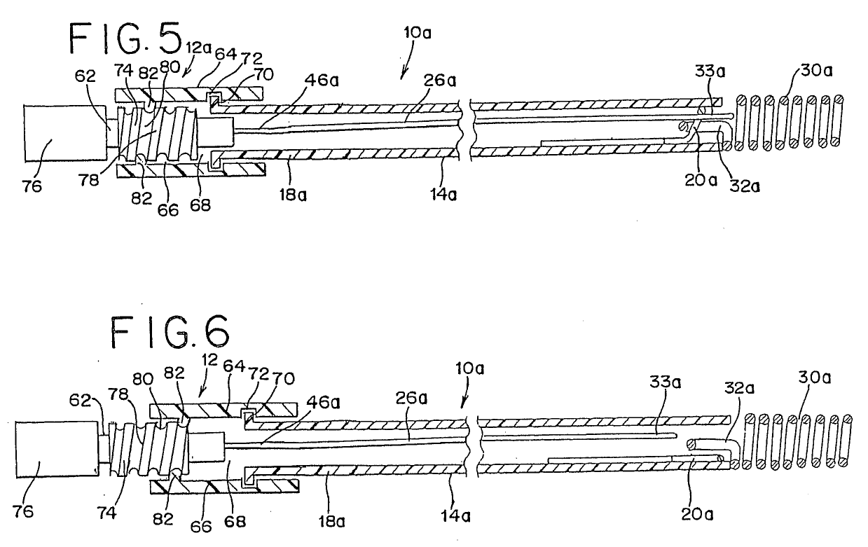

FIGS. 5 and 6 illustrate an alternative embodiment of a handle in accordance with present invention. In this embodiment, the deployment system 10a includes a handle 12a which includes a handle body 62 and a rotatable member 64. The rotatable member 64 is rotatably coupled to the proximal end portion 18a of the carrier member 14a so that the rotatable member is rotatable relative to the carrier member 14a and the handle body 62, as will be explained in more detail herein. The rotatable member 64 may be rotatably coupled to the proximal end portion 18a of the carrier member 14a by any suitable rotatable coupling configuration; for example, one suitable rotational coupling is a rotatable coupling similar to the rotatable couplings used in rotatable hemostatic valves, which are well known in the art.

In the illustrated embodiment, the rotatable member 64 includes an internal threaded surface 66 which defines a lumen 68. The proximal end portion 18a of the carrier member 14a is located within the lumen 68, and a rim 70 extending radially form the proximal end portion 18a of the carrier member 14a is located within a groove 72 of the rotatable member 64 to mechanically and rotatably connect the rotatable member 64 to the carrier member 14a.

The handle body 62 includes a threaded portion 74 and a gripping portion 76 extending from the threaded portion 74. The threaded portion 74 is located within the lumen 68 of the rotatable member 64 and includes a threaded surface 78 corresponding to and engaging the threaded internal surface 66 of the rotatable member 64. The threaded engagement between the threaded portion 74 of the handle body 62 and the internal threaded surface 66 of the rotatable member 64 may be any suitable threaded engagement. In the illustrated embodiment, the threaded surface 78 of the threaded portion 74 includes a grooved thread 80, and the threaded surface 66 of the rotatable member 64 includes at least one projection 82 that follows along the groove 80 as the hand body 62 and rotatable member 64 are rotated relative to one another.

When the handle body 62 and the rotatable member 64 are rotated relative to one another, the threaded engagement between the handle body 62 and the rotatable member 64 causes the handle body 62 to move axially in a proximal or distal direction depending on the direction of relative rotation and the desired use. The proximal end 46a of the control member 26a is operatively connected to the handle body 62 so that the control member 26a moves proximally and distally with the handle body 62.

The handle body 62 and rotatable member 64 may be rotated relative to one another by a variety of methods. For example, the gripping portion 76 of the handle body 62 may be grasped to hold the handle body 62 in a rotationally stationary position, and the rotatable member 64 may be rotated relative to the handle body 62. In another method, the rotatable member 64 may be held in a rotationally stationary position, and the handle body 62 may be rotated relative to the rotatable member 64. Further, the rotatable member 64 could be rotated in one direction and the handle body 62 could be rotated in the other direction.

As illustrated in FIG. 6, when the handle body 62 is rotated relative to the rotatable member 64, by any of the methods discussed above, the handle body 62 moves proximally or distally relative to the carrier member 14a. In the illustrated embodiment of the a deployment system, movement of the handle body 62 in the proximal direction causes the control member 26a, which is attached to the handle body, to also move in the proximal direction so that the distal end portion 33a of the control member 26a disengages engagement element 20a, thereby releasing medical device 30a in a similar manner as described above.

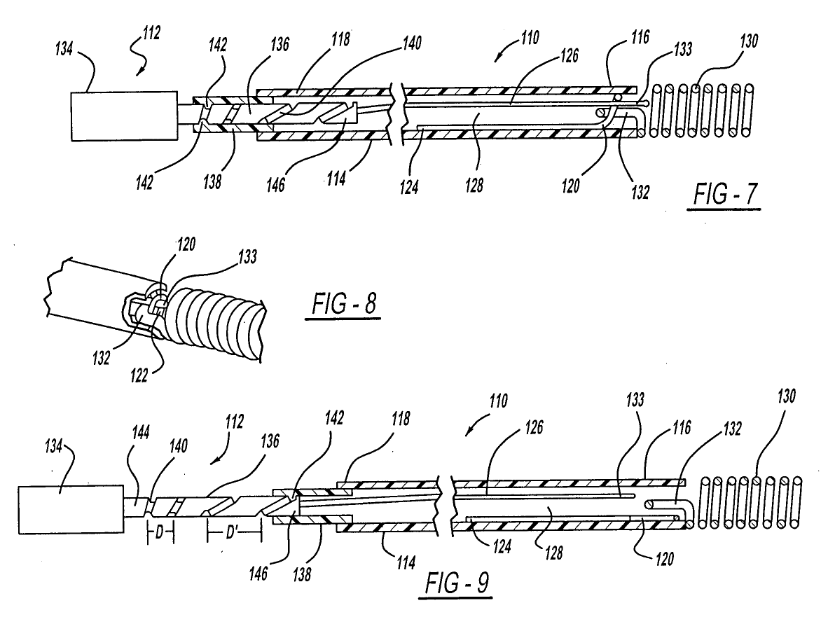

In another embodiment illustrated in FIGS. 7-9, a deployment system 110 further includes a control member 126, such as a control wire or pull wire, received within the lumen 128 of the carrier member 114 and operatively connected to the actuator 112. The control member 126 may be a wire comprised of any of a number of materials, including nitinol, and preferably, is sufficiently stiff to be advanced and/or retracted within the lumen 128 of the carrier member 114. The function of the control member 126 will be described in greater detail herein.

The actuator 112 is moveably connected to the proximal end portion 118 of the carrier member 114 and can move axially in a proximal direction from the position shown in FIG. 7 to the position shown in FIG. 8. When desired, for example in a deployment system in which the release of the medical device is caused by movement of the actuator 112 in a distal direction, the actuator 112 can also move axially in a distal direction from the position shown in FIG. 8 to the position shown in FIG. 7.

The actuator 112 includes a gripping portion 134 and a threaded portion 136. The gripping portion 134 can be configured to be gasped by hand, medical instrument or both. Preferably, the gripping portion 134 functions as a percutaneous handle and has a gripping surface, such as a knarled surface or protruding wings.

The threaded portion 136 of the actuator 112 is configured to threadable engage a corresponding threaded portion 138 of the proximal end portion 118 of the carrier member 114. The threaded engagement between the actuator 112 and the carrier member 114 can be any suitable threaded connection. In the illustrated embodiment, the threading of the threaded portion 136 of the actuator 112 comprises a groove 40 and the threading of the corresponding threaded portion 138 of the proximal end portion 118 of the carrier member 114 comprises one or protrusions 142 which engage and follow the groove 140 as the actuator 112 is rotated relative to the carrier member 114. In an alternative embodiment, the outer surface of the proximal end 18 of the carrier member 114 could include threading in the form of a groove, and the threaded portion of the actuator 112 could include a protrusion that engages and follows the groove.

When the actuator 112 is threadably engaged with the carrier member 114, rotation of the actuator relative to the carrier member 114 causes the actuator 112 to move proximally or distally in an axial direction depending on the direction of relative rotation between the actuator and carrier member.

Referring to FIG. 9, the grooved threading 140 located on the actuator 112 of this embodiment varies in pitch and has at least two different pitch sizes. For example, the pitch can have a first size equal to the distance D near or associated with the proximal end 144 of the threaded portion 136, and a second size equal to the distance D′ near or associated with the distal end 146 of the threaded portion 136. In the illustrated embodiment, the first size of the pitch (distance D) is shorter than the second size of the pitch (distance D′). In other words, the pitch is fine at or near the proximal end 144 and coarse at or near the distal end 146. However, depending on the desired use, the first pitch size (distance D) could be larger than the second pitch size (distance D′). Alternatively, the pitch could continually increase or decrease from the proximal end 144 to the distal end 146 of the threaded portion 136 of actuator 112.

Referring to FIG. 7, when the actuator 112 is rotated or unscrewed from the proximal end 118 of the carrier member 114, the projection 142 of threaded portion 138 of the carrier member 114 follows along the groove 40 and causes the actuator 112 to move axially in a proximal direction relative to the carrier member 114. As the projection 142 follows along the section of the groove 140 having a pitch size of D, the actuator 112 will have a first rate of axial movement relative to the carrier member 114. As actuator 112 is further rotated and the projection 142 follows along the section of the groove 140 having a pitch size of D′, the actuator 112 will have a second rate of axial movement relative to the carrier member which is faster than the first rate of axial movement. Thus, the threaded connection between the threaded portion 36 of the actuator 112 and the threaded portion 40 of the proximal end portion 118 of the carrier member 114 functions as a regulator that changes the rate of axial movement of the actuator relative to the carrier member.

The rate of axial movement of actuator 112 can be controlled by the speed at which the actuator is rotated relative to the carrier member and the size of the pitch of the threading of the actuator. For example, at a constant speed of actuator rotation relative to the carrier member, a smaller or finer pitch size will result in relatively slower axial movement of the actuator relative to the carrier member, and a larger or coarser pitch size will result in relatively faster axial movement of the actuator relative to the carrier member. Thus, the size of the pitch can be tailored to the desired use.

As discussed above, the control member 126 is connected to the actuator 112. Accordingly, as the actuator 112 is moved axially relative to the carrier member 114, the control member 126 is also moved axially, in the same direction and at the same rate as the actuator 112, relative to the carrier member 14. Therefore, in the illustrated deployment system 110 of FIG. 7, when the actuator 112 is rotated, the actuator 112 and the control member 126, which is connected to the actuator, move in a proximal direction relative to carrier member 114 and the engagement member 120. As explained above, the initial axial movement of the actuator 112 and the control member 126 is relatively slow, and as actuator 112 is further rotated, further movement in the axial direction is accelerated as compared to the initial movement. As will be appreciated from the above description, the initial relatively slow axial movement of the actuator 112 results in a slow tensioning of the control member which reduces the risk of breaking or snapping the control member during operation.

Referring to FIG. 9, and continuing with the same rotational direction noted immediately above, the control member 126 is moved proximally relative to the engagement member 120. In the illustrated detachment system for the medical device that is shown in the drawings, this proximal movement proceeds so that the distal end portion 133 of the control member 126 moves out of the opening 122 of the engagement member 120. Once the distal end portion 133 of the control member 126 is moved out of opening 122, the unconstrained engagement member 120 is free to allow release of the medical device. FIG. 9 shows the engagement returned to or moved to, its flat configuration, thereby releasing the medical device 130. This return can be due to a bias in the engagement member 120 or by a straightening-type of engagement with the medical device 130 as it separates from the detachment device.

When the engaged device 130 has been properly positioned and oriented, the actuator 112 is grasped by hand or instrument at the gripping portion 134 and rotated relative to the carrier member 114. As the actuator 112 is rotated, the actuator and control member 126 initially move relatively slowly in a proximal direction as a result of the fine or short pitch size associated with the proximal end portion 144 of the threaded portion 136 of the actuator 112. As the actuator 112 is further rotated, the actuator 112 and control member 126 move proximally at a more accelerated rate, as a result of the coarse or larger pitch distance associated with the distal end portion 144 of the threaded portions 136 of the actuator 112. Referring to FIG. 9, as the control member 126 moves in a proximal direction, it comes out of engagement with the engagement member 120. The engagement member 120 is then allowed to return to its original substantially flat condition, or is moved to a release condition by engagement with another component of the system, thereby releasing the aperture-containing end portion 132 of the implantable medical device 130 and deploying the medical device 130. Release also can be achieved by or be facilitated by opposing relative movement between the engagement member 120 and the medical device 130. The control member 126 may be provided with a radiopaque portion to provide visual feedback to indicate when the device 130 has been released.

It will be understood that the embodiments of the present invention which have been described are illustrative of some of the applications of the principles of the present invention. Numerous modifications may be made by those skilled in the art without departing from the true spirit and scope of the invention, including those combinations of features that are individually disclosed or claimed herein.

What is claimed is:

1. A deployment system for delivering an implantable medical device to a target location of a body vessel, comprising:
a generally elongated carrier member having a proximal end portion and a distal end portion;
an implantable medical device releasably attached to the distal end portion of the carrier member;
a control member whose movement causes the release of the implantable medical device from the distal end portion of the carrier member;
a handle having a rotatable member and a handle body, said rotatable member being rotationally coupled to the distal end portion of the carrier member, said rotatable member including an internal threaded surface defining a lumen;
the handle body having a threaded portion, said threaded portion of the handle body located within the lumen of the rotatable member and being threadably engaged with the internal threaded surface of the rotatable member, the handle body being operatively connected to the control member so that the control member moves with the handle body; and
wherein either the rotatable member or the handle body is rotated to cause the handle body to move in an axial direction relative to the carrier member, thereby causing movement of the control member to release the medical device.

2. The deployment system of claim 1 in which the threaded portion of the handle body includes a grooved thread, and in which the internal threaded surface of the rotatable member includes at least one projection that follows along the grooved thread of the threaded portion of the handle body.

3. The deployment system of claim 1 in which the handle body includes a gripping portion extending from the threaded portion.

4. The deployment system of claim 1 in which the relative rotational movement between the handle body and the rotatable member causes the handle body to move in a proximal or distal direction relative to the carrier member.

5. The deployment system of claim 1 in which the control member is a wire.

6. The deployment system of claim 1, in which said threaded portion of the handle body includes a pitch that has at least a first pitch size and a second pitch size, said first and second pitch sizes being different from each other.

7. The deployment system of claim 1 in which the pitch causes rotational movement of the rotatable member to retract the control member from the medical device.

8. The actuator of claim 1 in which the pitch causes rotational movement of the rotatable member to advance the control member toward the medical device.

9. The deployment system of claim 1 in which the threaded portion of the handle body has a proximal end portion and a distal end portion, the first pitch size is associated with proximal end portion of the threaded portion, and the second pitch size is associated with the distal end portion of the threaded portion, and said first pitch size is smaller than said second pitch size.

10. The deployment system of claim 1 in which the threaded portion of the handle body has a proximal end portion and a distal end portion, the first pitch size is associated with the proximal end portion of the threaded portion, and the second pitch size is associated with the distal end portion of the threaded portion, and said first pitch size is larger than said second pitch size.

11. The deployment system of claim 1 in which the threaded portion of the handle body has a proximal end portion and a distal end portion, and the pitch size continually changes from the proximal end portion to the distal end portion of the threaded portion.

12. A deployment system for delivering an implantable medical device to a target location of a body vessel, comprising:
a generally elongated carrier member having a proximal end portion and a distal end portion;
an implantable medical device releasably attached to the distal end portion of the carrier member; a control member whose movement causes the release of the implantable medical device from the distal end portion of the carrier member;
an actuator including a threaded portion that is threadably connected to a corresponding threaded portion of the carrier member located at the proximal end portion of the carrier member, and the control member being connected to said actuator, whereby rotational movement of the actuator causes the actuator to move in an axial direction relative to the carrier member, thereby causing movement of the control member in an axial direction to release the medical device from the distal end portion of the carrier member; and
one of said threaded portion of the actuator and said threaded portion of said carrier member including a pitch that has at least a first pitch size and a second pitch size, said first pitch size and said second pitch size being different from each other.

13. The deployment system of claim 12 in which the pitch causes rotational movement of the actuator to retract the actuator from the deployment device.

14. The deployment system of claim 12 in which the threaded portion of the actuator has a proximal end portion and a distal end portion, the first pitch size is associated with proximal end portion of the threaded portion, and the second pitch size is associated with the distal end portion of the threaded portion, and said first pitch size is smaller than said second pitch size.

15. The deployment system of claim 12 in which the threaded portion of the actuator has a proximal end portion and a distal end portion, the first pitch size is associated with the proximal end portion of the threaded portion, and the second pitch size is associated with the distal end portion of the threaded portion, and said first pitch size is larger than said second pitch size.

16. The deployment system of claim 12 in which the threaded portion of the actuator has a proximal end portion and a distal end portion, and the pitch size continually changes from the proximal end portion of the threaded portion to the distal end portion of the threaded portion.

17. A deployment system for delivering an implantable medical device to a target location of a body vessel, comprising:
a generally elongated carrier member having a proximal end portion and a distal end portion;
an implantable medical device releasably attached to the distal end portion of the carrier member; a control member whose movement causes the release of the implantable medical device from the distal end portion of the carrier member;
an actuator movably connected to the proximal end portion of the carrier member, said actuator moveable in an axial direction relative to the carrier member, and the control member being connected to the actuator, whereby movement of the actuator relative to the carrier member causes movement of the control member to release the medical device from the distal end portion of the carrier member; and
a regulator that changes the rate of relative axial movement between the actuator and the carrier member.

18. The deployment system of claim 17 in which the regulator is comprised of a threaded portion of the actuator and a corresponding threaded portion of the carrier member, and at least one of said threaded portion of the actuator and threaded portion of the carrier member includes a pitch having at least a first pitch size and a second pitch size that varies from said first pitch size.

19. The deployment system of claim 17 in which the threaded portion of the actuator has a proximal end portion and a distal end portion, the proximal end portion of the threaded portion has a first pitch size and the distal end portion of the threaded portion has a second pitch size, and said first pitch size is smaller than said second pitch size.

20. The deployment system of claim 17 in which the threaded portion of the actuator has a proximal end portion and a distal end portion, the proximal end portion of the threaded portion has a first pitch size and the distal end portion of the threaded portion has a second pitch size, and said first pitch size is larger than said second pitch size.
